(19) United States
(12) Patent Application Publication
(10) Pub. No.: US 2010/0099674 A1
(43) Pub. Date: Apr. 22, 2010

(54) METHOD AND MEANS FOR PREVENTING AND INHIBITING TYPE III SECRETION IN INFECTIONS CAUSED BY GRAM-NEGATIVE BACTERIA

(75) Inventor: Mikael Elofsson, Umea (SE)

Correspondence Address: DICKSTEIN SHAPIRO LLP, 1633 Broadway, NEW YORK, NY, 10019, US

(73) Assignee: Innate Pharmaceuticals AB, Umea (SE)

(21) Appl. No.: 12/531,397

(22) PCT Filed: Mar. 18, 2008
(86) PCT No.: PCT/SE2008/000199

§ 371 Date: Sep. 15, 2009

(30) Foreign Application Priority Data
Mar. 21, 2007 (SE) 0700695 0
Jun. 21, 2007 (SE) 0701509 2

(51) Int. Cl. A61K 31/5377; C07D 401/06; C07D 413/06; C07D 215/38; A61K 31/497; A61K 31/4709; A61P 31/04

(52) U.S. Cl. 514/235.2; 544/363; 544/128; 546/179; 514/253.06; 514/314

(57) ABSTRACT

A means for decreasing bacterial virulence in a mammal including man or in a plant by inhibition of the Type III secretion system at concentrations that do not prevent or substantially reduce bacterial growth comprises an N-substituted 7-quinolylmethyl amine, in particular one that is further substituted in 5- and 8-position on the quinoline ring. A corresponding therapeutic method and a pharmaceutical composition are also disclosed.

### FIELD OF THE INVENTION

The invention relates to a method and a means for preventing and inhibiting Type III secretion in infections caused by Gram-negative bacteria.

### BACKGROUND OF THE INVENTION

Antibiotic resistance among micro-organisms has dramatically escalated in the last ten years.¹⁻² Such resistance proliferates readily in the bacterial kingdom through gene transfer, making the spread of resistance hard to control. A multitude of inefficient efforts to improve the situation have made it clear that development of novel anti-microbial drugs rapidly must take place. Recent advances in combinatorial chemistry,³⁻⁵ genomics,^{6, 7} and screening technologies^{8, 9} have increased the capacity to identify novel bacterial targets and compounds that interfere with them. A novel mode of action is that of targeting microbial virulence rather than growth.^{10, 11} Virulence includes events that enable the bacterium to enter the host, disarm the host's defense, multiply, and finally spread within the host or to a new host. Agents that target virulence are potentially effective antimicrobials but also apply less selective pressure for resistance. Recent studies have revealed that various pathogenic bacteria use related virulence systems, findings that contradict the long held paradigm that each bacterium has a unique mode of action. The virulence system can be attacked by potentially effective antimicrobials derived by using a chemical genetics approach.

Regulation of the Type III secretion machinery for Yersinia pseudotuberculosis is relatively well understood.^{12, 13} Many of the genes required for the Type III secretion system are carried on a 70-kbp plasmid. Most of the genes have one of three designations; Ysc (Yersinia secretion proteins), Yops (Yersinia outer proteins) or Sycs (specific yop chaperones). The machinery can be compared to a syringe that injects effector proteins from the bacteria directly into the eukaryotic cell through pores in the membranes. The Type III secretion system (T3SS) is not constantly produced; it is only when the bacteria enter a host that genes from the plasmid are expressed. The regulation is not fully understood, but different parts of the machinery are produced in response to different signals, FIG. 1. The core structure is assembled when bacteria enter a host and the temperature shifts to 37° C., regulated by the temperature-triggered protein LcrF. Another signal is the contact with beta-integrin on the eukaryotic target cell that triggers the secretion of the inhibitor LcrQ. This secretion results in a strong Yop-production. Production and secretion of Yop has been found to be Ca²⁺ dependent in vitro. In the luciferase assay described in WO 2004/022775, a luxAB gene is under the control of a YopE-promoter, allowing identifying compounds that interfere with the type III secretion machinery in the absence of eukaryotic cells. Transcription of luxAB results in a luciferase that catalyses oxidation of flavine mononucleotides in the presence of an aldehyde, which is a light-emitting reaction. Secretion of Yops is the result of a fully functional T3SS and if any part of the complex T3SS is affected, a reduced transcription of yopE/luxAB will be seen. The assay includes a bacteria strain comprising a luxAB construct, grown in Ca²⁺ depleted medium, incubating the bacteria in Ca²⁺ depleted medium at 37° C. with an agent which antibacterial effect shall be determined, recording the light emitted by the bacteria upon addition of decanal. This luciferase assay is well suited for high-throughput screening of large compound collections.¹⁴ Compounds that interfere with the secretion system have potential both as drug candidates and molecular probes for functional studies of the T3SS in Yersinia and other Gram-negative.¹⁵⁻¹⁷ Furthermore, a powerful feature of this assay is that it also can be used to identify activators of the system. Such compounds will be useful for investigations aimed at understanding the regulation of the system. Screening of a 17,500 compound library by this assay resulted in a number of hits, which inhibit the Type III secretion system at concentrations that do not prevent bacterial growth.

### OBJECTS OF THE INVENTION

It is an object of the invention to provide a method and a means for preventing and inhibiting Type III secretion in infections caused by Gram-negative bacteria in a mammal or a plant.

It is another object of the invention to provide a means for preventing and/or inhibiting Type III secretion in infections caused by Shigella subspecies.

Further objects of the invention will become evident from the following summary of the invention, a number of preferred embodiments illustrated in figures, and the appended claims.

### SUMMARY OF THE INVENTION

According to the present invention is disclosed a method and a means for decreasing bacterial virulence in a mammal including man or in a plant by inhibition of the Type III secretion system at concentrations that do not prevent bacterial growth. The means is a pharmacologically active agent which is an N-substituted 7-quinolylmethyl amine, in particular one that is further substituted in 5- and 8-position on the quinoline ring.

According to a preferred aspect of the invention the methylene moiety of the N-substituted 7-quinolylmethyl amine is additionally substituted by any of methyl, ethyl, phenyl, chlorophenyl, bromophenyl, in particular 4-chlorophenyl.

According to a particularly preferred aspect of the invention the N-substituted 7-quinolylmethyl amine is N-substituted (8-hydroxy-7-quinolyl)methyl amine.

According to further preferred aspect of the invention is disclosed an agent capable of decreasing bacterial virulence in a mammal including man or in a plant by inhibition of the Type III secretion system, which is a quinoline of the general formula I

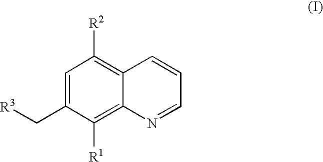

wherein
R¹ is OH, or C₁-C₄-alkoxy;
R² is H, nitro, Cl, Br, F or I;
R³ is N-morpholinyl, N-piperazinyl, N-pyrrolidinyl or NR⁴R⁵, wherein N-morpholinyl is optionally mono- or disubstituted in 3- or/and 5-position by same or different C₁-C₄-alkyl and wherein N-piperazinyl is 4-R⁶-1-N-piperazinyl, wherein R⁶ is phenyl, methyl, methoxyphenyl, fluorophenyl, chlorophenyl, pyridyl;
wherein N-pyrrolidinyl is optionally monosubstituted in 3-position, preferably by phenyl or phenyl monosubstituted by methoxy, fluoro, chloro, bromo;
R⁴ and R⁵ are both C₁-C₄-alkyl or one of them is C₁-C₄-alkyl and the other is cyclohexyl.

In the quinoline derivative of the general formula I it is more preferred for

R¹ to be OH.

In the quinoline derivative of the general formula I it is more preferred for
R³ to be N-morpholinyl, N-piperazinyl or N-pyrrolidinyl, wherein N-morpholinyl is optionally disubstituted in 3- and 5-position by C₁-C₄-alkyl, and N-pyrrolidinyl is monosubstituted in 3-position, preferably by phenyl or phenyl monosubstituted by methoxy, fluoro, chloro, bromo.

Preferred Type III secretion blocker agents of the general formula I are shown in FIG. 3.

According to the invention is also disclosed a pharmaceutical composition comprising a pharmacologically effective amount of the agent of the invention and an pharmaceutically acceptable excipient. Any type of pharmaceutical composition capable of providing a pharmacologically effective Type III secretion inhibiting plasma or local concentration of the agent of the invention, in particular plasma concentrations ranging from 0.001 μg/ml to 10 μg/ml and more, is comprised by the invention. Oral and parenteral administration is preferred but does not exclude other ways of administration. The pharmaceutical composition of the invention may furthermore comprise an adjuvant enhancing the uptake of the Type III secretion blocker agent by infected cells or the attachment to such cells.

The method of treating infection by Gram-negative bacteria, in particular by a Shigella subspecies, in a person or an animal or a plant comprises the administration of the agent of the invention or the pharmaceutical composition of the invention to said person or animal or plant.

The invention will now be described in more detail by reference to preferred but not limiting embodiments illustrated in a number of figures.

### SHORT DESCRIPTION OF THE FIGURES

FIG. 1 shows the current model for regulation of Yop and Syc expression; (A) At 37° C. LcrF is a positive regulator of Syc and Yop expression but at this stage Yop expression is suppressed by the negative regulator LcrQ; (B) Upon eukaryotic cell contact or Ca²⁺ depletion at 37° C. LcrQ is secreted resulting in Yop expression; (C) Schematic representation of reporter gene constructs for identification of inhibitors or activators of Yersinia Type III secretion system in absence of eukaryotic target cells;

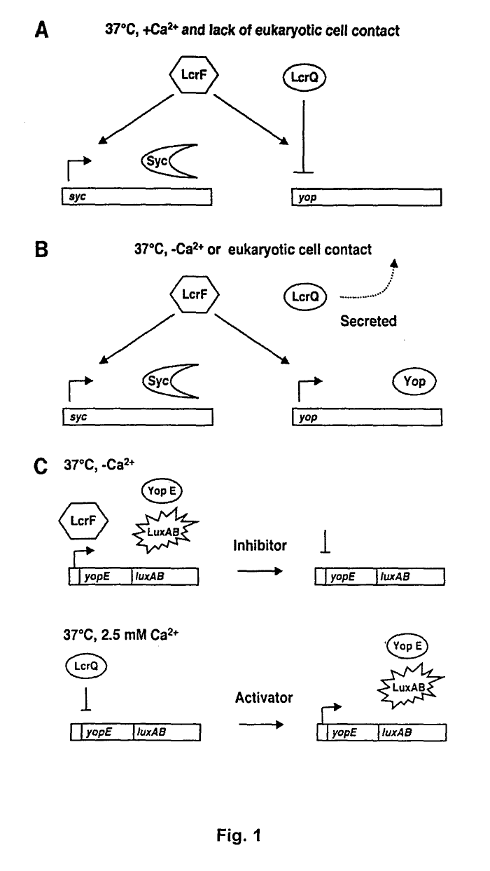

FIG. 2 shows an ex vivo test method involving CalceinAM uptake by living cells with conversion to green fluorescent Calcein and Sytox Orange uptake by cells with damaged cell membrane with red fluorescence after bonding to the cell DNA;

FIG. 3 shows preferred Type III secretion blocker agents of the invention.

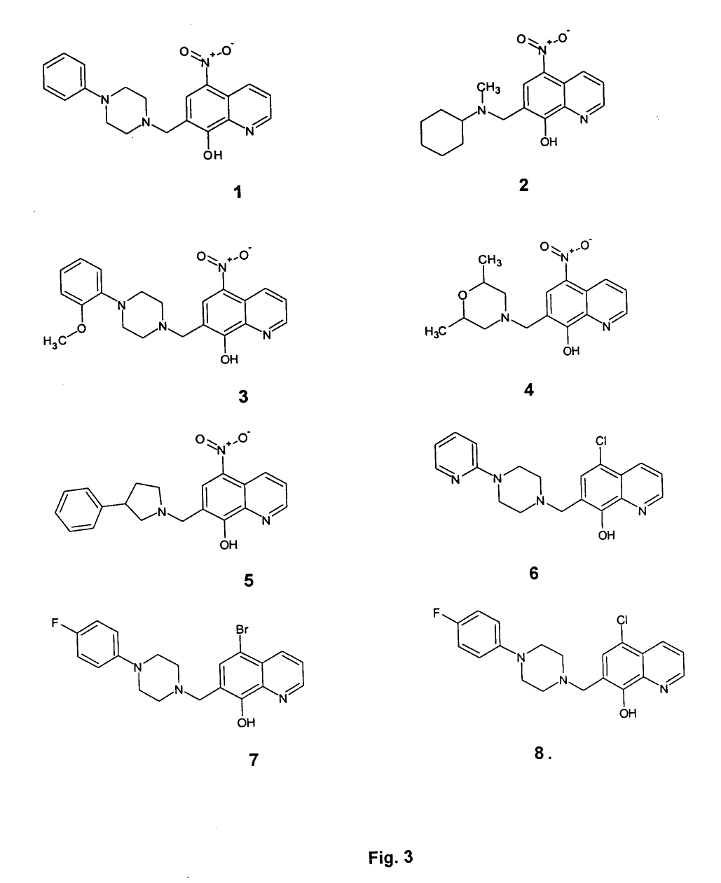

### DESCRIPTION OF PREFERRED EMBODIMENTS

Bacterial strains. All strains used were Y. psedotuberculosis serotype III (YPIII) and in the following text strains are only labelled with the name of the virulence plasmid, deposited May 22, 2007 at the Polish Collection of Microorganisms (PCM), Accession Nos. B/00014, B/00015, and B/00016 (given by the International Depositary Authority).

Compounds. The screen was performed on a chemical library consisting of 17,500 unique compounds in 96-well plate format from ChemBridge Corporation (16981 Via Tazon, Suite G San Diego, Calif. 92127, USA). The compounds were dissolved in DMSO to give a stock solution of 5 mM. For compounds further characterised in the described experiments, additional 5 or 10 mg samples were purchased from ChemBridge Corp.

Luciferase Assay. The assay, described in WO 2004/022775, measures the effect of a substance in Yersinia strain pIB102EL where a YopE gene is transcriptionally fused to the luxAB gene. By growing the bacteria at 37° C. in a medium depleted for Ca²⁺, YopE and luxAB expression are induced. The expression of luxAB can be measured as light emission by adding n-decanal to the solution. The blocking effect of a substance can thus be seen as a decreased light emission. The measurement is performed in white 96-well plates in a microplate reader. Strain with the luxAB construct was prepared from the yadA mutant pIB102 by constructing yopE-luxAB operon fusions essentially as described.¹⁸ The resulting strain pIB102EL was struck and grown at room temperature on LB-plates containing chloramphenicol (Sigma) at a final concentration of 25 μg/mL. From plates not older than one week, bacteria for experiments were grown in liquid brain/heart infusion broth (Oxoid; Unipath Ltd., Basington, UK) containing 2.5 mM CaCl₂ or 20 mM MgCl₂ and 5 mM EGTA for Ca²⁺ depletion.

YopH Assay. The assay, described by F. Liang et al., 19 comprises detection of secretion of one of the effector molecules (YopH), in which the ability of YopH to function as a protein tyrosine phosphatase was utilised. Yersinia-strain pIB102 was induced for Type III secretion by depleting Ca²⁺ from the growth medium at 37° C. The activity of YopH was measured by investigating dephosphorylation by YopH of the chromogenic substrate pNPP (p-nitrophenyl phosphate) into the intensely yellow p-nitrophenol. Dephosphorylated pNPP, i.e. p-nitrophenol, was measured in a microplate reader at 405 nm. The blocking effect of a substance was seen as a decreased dephosphorylation and thus decreased colour emission of pNPP when different concentrations of the substances (0, 10, 20, 50 and 100 μM) were added to the growth medium to a final volume 100 μl.

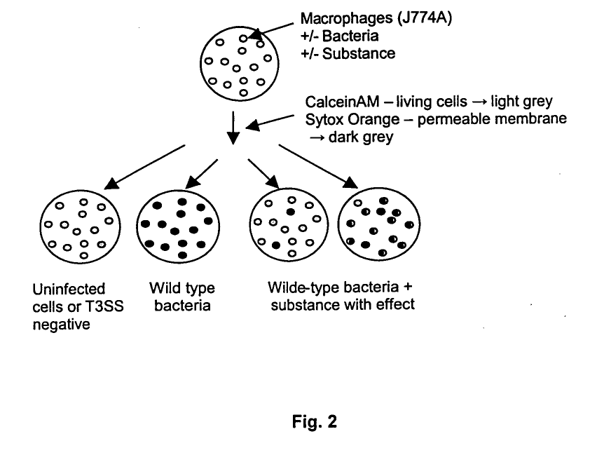

Inhibition Test—ex vivo test. The test, described by Bailey et al.,¹⁴ comprises J774A cells (Deutsche Sammlung von Mikroorganismen and Zellkulturen GmbH) seeded into a 96-well plate (7×10⁴ cells/well) in DMEM (Dulbecco's Modified Eagle's Medium, Gibco) with 10% FCS (Fetal Calf Serum) and gentamicin (3 μg/ml), grown for 12-24 h at 37° C. in 5% CO₂ Y. pseudotuberculosis YPIII (pIB102)²⁰ and the translocation-deficient mutant, yopB (YPIII (pIB604)²¹) strains were grown over night at 26° C. in LB-broth supplemented with 25 μg/ml kanamycin, diluted 1/10 in DMEM and incubated on a rotary shaker at 26° C. for 1 h followed by 2 h at 37° C. prior to infection. The J774A cells were washed once with PBS and 50 μl fresh DMEM (without FCS and without gentamicin) containing the different compounds and 50 μl of T3SS induced Y. pseudotuberculosis (OD₆₀₀=0.002) was added, giving a final multiplicity of infection (MOI) of 10. After 16 h of infection CalceinAM (Molecular Probes; Invitrogen) and Sytox Orange (Molecular Probes; Invitrogen) in 20 μl PBS were added to a final concentration of 1 μM and 0.05 μM respectively and the plate was incubated for 40 min at 37° C. in 5% CO₂. CalceinAM is enzymatically transformed to green fluorescent Calcein in healthy cells (LIVE/DEAD® Viability/Cytotoxicity Kit *for mammalian cells*, Invitrogen). If the cell membrane of the eukaryotic cells was damaged, Sytox Orange was taken up and bound to the DNA which increased the red florescence of Sytox Orange 400 times (FIG. 2). The fluorescence was then read in a microplate reader (TECAN GENios, Tecan Trading AG, Switzerland; Calcein at 485/535 nm and Sytox Orange at 535/595 nm) and the results were confirmed using fluorescent microscopy. Each compound was tested at least twice in triplicates in this assay. From the ex vivo experiments the toxicity of the substances was estimated; if a substance is toxic to the cells the relative amount of Calcein decreases and the relative amount of Sytox Orange increases without addition of pIB102. Substances toxic to eukaryotic cells were discarded and not tested further.

ETEC Negative Control. Enterotoxic Escherichia coli (ETEC) was used as a negative control in the ex vivo test, since this bacterium is a Gram-negative pathogen bacterium causing diarrhea but while lacking the Type III secretion system. The perfect T3SS inhibiting substance should not have effect on ETEC in ex vivo infection studies. In reality, T3SS inhibiting substances at higher concentrations might affect ETEC even though they should be T3SS specific since they might be toxic at high concentration.

TABLE 1
Inhibition tests performed on Y. pseudotuberculosis
with the Type III secretion blocker agents of the invention.

| | | | Ex vivo | |
|---|---|---|---|---|
| | Lux | | | ETEC |
| Substance | Assay* | YopH Assay* | Y. pseudotb.† | (Neg Control)‡ |
| 1 | ++ | ++ | ++ | No data |
| 2 | + | − | − | − |
| 3 | ++ | + | + | − |
| 4 | + | + | + | + |
| 5 | ++ | ++ | + | ++ |
| 6 | + | + | + | + |
| 7 | + | + | ++ | ++ |
| 8 | + | + | ++ | + |
| Other | − | No data | No data | No data |
| analogues | | | | |
| tested | | | | |

*+ = <50% reduction of assay read-out at 50 μM; ++ = <50% reduction of assay read-out at 20 μM;
†+ = >70% survival of the infected cells at 20 μM; ++ = >70% survival of the infected cells at 10 μM;
‡+ = >70% survival of the infected cells at 50 μM; ++ = >70% survival of the infected cells at 20 μM

### REFERENCES

1. Finch, R. G. Antibiotic resistance. J. Antimicrobial Chemother. 42, 125-128 (1998).
2. Chin, G. J., Marx, J. & Eds. Resistance to antibiotics. Science 264, 359-393 (1994).
3. An, H. & Cook, P. D. Methodologies for generating solution-phase combinatorial libraries. Chem. Rev. 100, 3311-3340 (2000).
4. Cork, D. & Hird, N. Work-up strategies for high-throughput solution synthesis. Drug Discovery Today 7, 56-63 (2002).
5. Lou, B. Novel strategies for solid-phase construction of small-molecule combinatorial libraries. Drug Discovery Today 6, 1288-1294 (2001).
6. Buysse, J. M. The role of genomics in antibacterial target discovery. Curr. Med. Chem. 8, 1713-1726 (2001).
7. McDevitt, D. & Rosenberg, M. Exploiting genomics to discover new antibiotics. Trends Microbiol. 9, 611-617 (2001).
8. Sundberg, S. A. High-throughput and ultra-high throughput screening: solution- and cell-based approaches. Curr. Opin. Biotechnol. 11, 47-53 (2000).
9. Hertzberg, R. P. & Pope, A. J. High-throughput screening: new technology for the 21st century. Curr. Opin. Chem. Biol. 4, 445-451 (2000).
10. Goldschmidt, R. M., Macielag, M., Hlasta, D. J. & Barrett, J. F. Inhibition of virulence factors in bacteria. Curr. Pharm. Design 3, 125-142 (1997).
11. Alksne, L. E. & Projan, S. J. Bacterial virulence as target for antimicrobial chemotherapy. Curr. Opin. Biotechnol. 11, 625-636 (2000).
12. Cornelis, G. R. & Wolf-Watz, H. The Yersinia Yop virulon: A bacterial system for subverting eukaryotic cells. Mol. Microbiol. 23, 861-867 (1997).
13. Cornelis, G. R. Molecular and cell biology aspects of plague. Proc. Nat. Acad. Sci. USA 97, 8778-8783 (2000).
14. Kauppi, A. M., Nordfelth, R., Hägglund, U., Wolf-Watz, H. & Elofsson, M. Salicylanilides are potent inhibitors of type III secretion in Yersinia. Adv. Exp. Med. Biol. 529, 97-100 (2003).
15. Nordfelth, R., Kauppi, A. M., Norberg, H. A., Wolf-Watz, H. & Elofsson, M. Small molecule inhibitors specifically targeting type III secretion. Infection & Immunity 73, 3104-3114 (2005).
16. Muschiol, S., Bailey, L., Gylfe, Å., Sundin, C., Hultenby, K., Bergström, S., Elofsson, M., Wolf-Watz, H., Normark, S., & Henriques-Normark, B. A small-molecule inhibitor of type III secretion inhibits different stages of the infectious cycle of Chlamydia trachomatis. Proc. Natl. Acad. Sci. 103(39), 14566-71 (2006).
17. Bailey, L., Gylfe, Å., Sundin, C., Muschiol, S., Elofsson, M., Nordström, P., Henriques-Normark, B., Lugert, R., Waldenström, A., Wolf-Watz, H. & Bergström, S. Small molecule inhibitors of Type III secretion in Yersinia block the Chlamydia pneumoniae infection cycle. FEBS Letters In press, (2007).
18. Forsberg, Å. & Rosqvist, R. In vivo expression of virulence genes of Yersinia pseudotuberculosis. Infect. Agents Dis. 2(4), 257-8 (1993).
19. Liang, F., Huang, Z., Lee, S. Y., Liang, J., Ivanov, M. I., Alonso, A., Bliska, J. B., Lawrence, D. S., Mustelin, T. & Zhang, Z. Y. Aurintricarboxylic acid blocks in vitro and in vivo activity of YopH, an essential virulent factor of Yersinia pestis, the agent of plague. J. Biol. Chem. 278, 41734-41 (2003).
20. Bolin, I. & Wolf-Watz, H. Molecular cloning of the temperature-inducible outer membrane protein 1 of Yersinia pseudotuberculosis. Infect. Immun. 43(1), 72-8 (1984).
21. Hakansson, S., Schesser, K., Persson, C., Galyov, E. E., Rosqvist, R., Homble, F. & Wolf-Watz, H. The YopB protein of Yersinia pseudotuberculosis is essential for the translocation of Yop effector proteins across the target cell plasma membrane and displays a contact-dependent membrane disrupting activity. EMBO J., 15(21), 5812-23 (1996).

1. An agent capable of decreasing bacterial virulence in a mammal including man or in a plant by inhibition of the Type III secretion system at concentrations that do not prevent or substantially reduce bacterial growth, which is an N-substituted 7-quinolylmethyl amine.

2. The agent of claim 1, wherein the methylene moiety of the N-substituted 7-quinolylmethyl amine is additionally substituted by methyl, ethyl, phenyl, chlorophenyl, or bromophenyl.

3. The agent of claim 1, wherein the N-substituted 7-quinolylmethyl amine is a N-substituted (8-hydroxy-7-quinolyl)methyl amine.

4-5. (canceled)

6. An agent capable of decreasing bacterial virulence in a mammal including man or in a plant by inhibition of the Type III secretion system, which is a quinoline of the general formula I 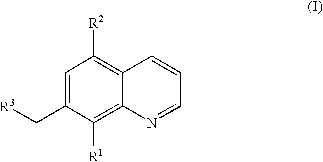
wherein
R¹ is OH, or Cᵢ-C₄-alkoxy;
R² is H, nitro, Cl, Br, F or I;
R³ is N-morpholinyl optionally mono- or disubstituted in the 3- or 5- or both 3- and 5-positions by the same or different C₁-C₄-alkyl, 4-R⁶-1-N-piperazinyl in which R⁶ is phenyl, methyl, methoxyphenyl, fluorophenyl, chlorophenyl, or pyridyl, N-pyrrolidnyl optionally monosubstituted in the 3-position, or NR⁴R⁵;
R⁴ and R⁵ are both C₁-C₄-alkyl or one of them is C₁-C₄-alkyl and the other is cyclohexyl.

7. The agent of claim 6, wherein R¹ is OH.

8. The agent of claim 6, wherein R³ is N-morpholinyl, N-piperazinyl or N-pyrrolidinyl, in which the N-morpholinyl is optionally disubstituted in 3- and 5-position by Ci-C₄-alkyl and the N-pyrrolidinyl is monosubstituted in 3-position by phenyl or phenyl monosubstituted by methoxy, fluoro, chloro, or bromo.

9. The agent of claim 1, wherein the Type III secretion blocker is chosen from

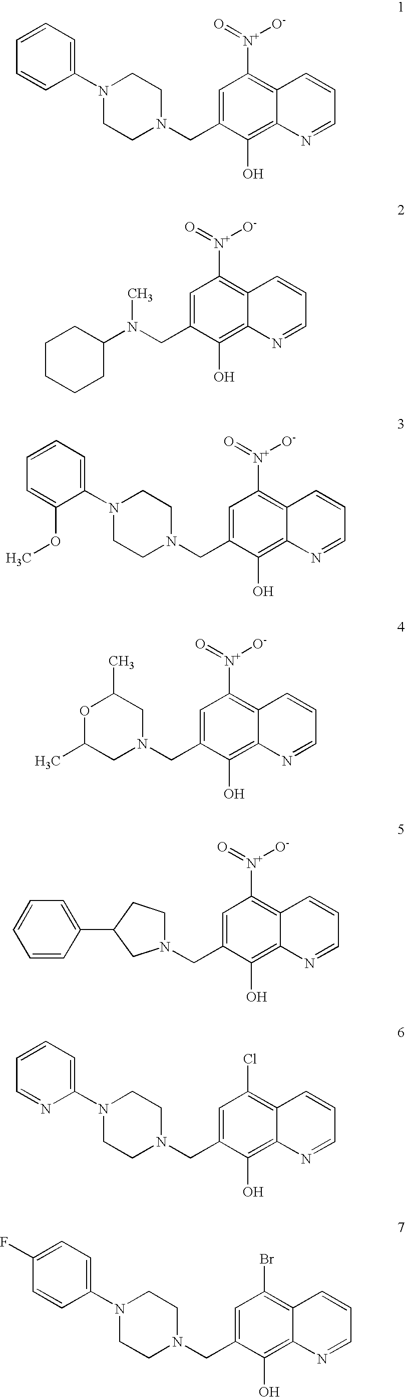

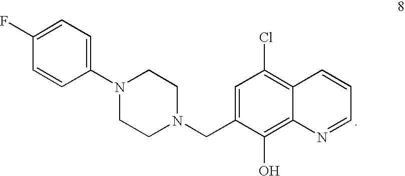

10. A method of preventing and/or inhibiting an infection caused by Gram-negative bacteria having a Type III secretion system, comprising the administration of an agent according to claim 1.

11. The method of claim 10, wherein the infection is caused by Shigella subspecies.

12. The method of claim 10, comprising repeated administration until said Gram-negative bacteria is no longer detectable in plasma or at site of infection.

13. The method of claim 10, wherein the administration extends over a period of at least one day.

14. A pharmaceutical composition for preventing and/or inhibiting Gram-negative bacteria, comprising a pharmacologically effective amount of the agent of claim 1 and a pharmaceutically acceptable carrier.

15. The composition of claim 14, in form of a single dose comprising from 0.001 μg/ml to 10 μg/ml.

16. The composition of claim 14, for oral administration.

17. The composition of claim 14, for parenteral administration.

18. The composition of claim 14, in a controlled-release form.

19. The agent of claim 1, wherein the amount is such that bacterial growth is not reduced by more than 20% at a therapeutically effective plasma or local concentration.

20. A method of treating infection by Gram-negative bacteria in a person or an animal or a plant, comprising the administration of the the pharmaceutical composition of claim 14.

21. The agent of claim 1, wherein the N-substituted 7-quinolylmethyl amine is additionally substituted at the 5- and 8-position on the quinoline ring.

22. The agent of claim 21, wherein the methylene moiety of the N-substituted 7-quinolylmethyl amine is additionally substituted by 4-chlorophenyl.
